(19) United States
(12) Patent Application Publication
(10) Pub. No.: US 2024/0398213 A1
(43) Pub. Date: Dec. 5, 2024

(54) ILLUMINATION METHOD, ILLUMINATION DEVICE, AND ENDOSCOPE SYSTEM

(71) Applicant: OLYMPUS CORPORATION, Tokyo (JP)

(72) Inventors: Mitsuru NAMIKI, Saitama (JP); Takehiro MIKI, Tokyo (JP); Atsuyoshi SHIMAMOTO, Tokyo (JP)

(73) Assignee: OLYMPUS CORPORATION, Tokyo (JP)

(21) Appl. No.: 18/807,153

(22) Filed: Aug. 16, 2024

Related U.S. Application Data
(63) Continuation of application No. PCT/JP2022/009133, filed on Mar. 3, 2022.

(51) Int. Cl. A61B 1/00

(52) U.S. Cl. CPC A61B 1/00165; A61B 1/00117

(57) ABSTRACT

An illumination method includes causing coherent light from a light source to enter a multimode propagation path via an incidence surface, relatively oscillating the light incident on the incidence surface and the incidence surface so as to temporally change at least one of an incidence position and an incidence angle of the light on the incidence surface, and radiating the light that has propagated through the propagation path onto a target.

### CROSS-REFERENCE TO RELATED APPLICATIONS

This is a continuation of International Application PCT/JP2022/009133 which is hereby incorporated by reference herein in its entirety.

### TECHNICAL FIELD

The present invention relates to illumination methods, illumination devices, endoscope systems, and endoscopes.

### BACKGROUND ART

In the related art, laser light sources are used in illumination devices (e.g., see Patent Literatures 1 to 3). A laser light source is advantageous over other types of light sources, such as a lamp light source or an LED, in having higher intensity and a narrower band. In detail, laser light is brighter than light from other light sources and can thus illuminate a subject more brightly. Furthermore, since the wavelength width of laser light is 1 nm or smaller, special-light observation, such as NBI (narrow band imaging), is possible without using an optical filter, such as a band-pass filter.

On the other hand, illumination using a laser light source is disadvantageous in that speckles may occur on the subject. Patent Literatures 1 and 2 each disclose a solution for reducing speckles by using a piezoelectric body or an air current to oscillate an intermediate position of an optical fiber that optically guides the laser light. Patent Literature 3 also discloses a solution for reducing speckles by rotating a light diffuser disposed between a focusing optical system and a collimator optical system.

### CITATION LIST

### Patent Literature

### {PTL 2}

Japanese Unexamined Patent Application, Publication No. 2010-172651

### {PTL 2}

Japanese Unexamined Patent Application, Publication No. 2018-117933

### {PTL 3}

The Publication of Japanese Patent No. 5682813

### SUMMARY OF THE INVENTION

An aspect of the present invention provides an illumination method including: causing coherent light from a light source to enter a multimode propagation path via an incidence surface; relatively oscillating the light incident on the incidence surface and the incidence surface so as to temporally change at least one of an incidence position and an incidence angle of the light on the incidence surface; and radiating the light that has propagated through the propagation path onto a target.

Another aspect of the present invention provides an illumination device including: a first optical guide member that optically guides coherent light from a light source; a second optical guide member that has an incidence surface, an output surface, and a multimode propagation path between the incidence surface and the output surface and that causes the light output from a distal end of the first optical guide member to enter the propagation path via the incidence surface; and an oscillation mechanism that relatively oscillates the light incident on the incidence surface and the incidence surface so as to temporally change at least one of an incidence position and an incidence angle of the light on the incidence surface.

Another aspect of the present invention provides an endoscope system including a light source device and an endoscope connected to the light source device. The light source device includes a light source, a first optical guide member that optically guides coherent light from the light source, and an oscillation mechanism. The endoscope includes a second optical guide member that has an incidence surface, an output surface, and a multimode propagation path between the incidence surface and the output surface and that causes the light output from a distal end of the first optical guide member to enter the propagation path via the incidence surface. The oscillation mechanism relatively oscillates the light incident on the incidence surface and the incidence surface so as to temporally change at least one of an incidence position and an incidence angle of the light on the incidence surface.

Another aspect of the present invention provides an endoscope including: a first optical guide member that optically guides coherent light from a light source; a second optical guide member that has an incidence surface, an output surface, and a multimode propagation path between the incidence surface and the output surface and that causes the light output from a distal end of the first optical guide member to enter the propagation path via the incidence surface; an oscillation mechanism that relatively oscillates the light incident on the incidence surface and the incidence surface so as to temporally change at least one of an incidence position and an incidence angle of the light on the incidence surface; and an imaging unit that captures an image of a target illuminated with the light output from the output surface of the second optical guide member.

### BRIEF DESCRIPTION OF THE DRAWINGS

FIG. 1 illustrates the overall configuration of an illumination device according to a first embodiment.

FIG. 2 illustrates light entering an incidence surface of a second optical guide member from an oscillating distal end of a first optical guide member and light propagating through the second optical guide member.

FIG. 3 is a flowchart illustrating an illumination method using an illumination device.

FIG. 4A illustrates the overall configuration of a modification of the illumination device in FIG. 1.

FIG. 4B illustrates the overall configuration of another modification of the illumination device in FIG. 1.

FIG. 4C illustrates the overall configuration of another modification of the illumination device in FIG. 1.

FIG. 4D illustrates the overall configuration of another modification of the illumination device in FIG. 1.

FIG. 4E illustrates the overall configuration of another modification of the illumination device in FIG. 1.

FIG. 4F illustrates the overall configuration of another modification of the illumination device in FIG. 1.

FIG. 5 illustrates the overall configuration of an illumination device according to a second embodiment.

FIG. 6A illustrates the overall configuration of a configuration example of an endoscope according to a third embodiment.

FIG. 6B illustrates the overall configuration of another configuration example of the endoscope according to the third embodiment.

FIG. 7A illustrates the overall configuration of an endoscope system according to a fourth embodiment.

FIG. 7B illustrates the overall configuration of a modification of the endoscope system in FIG. 7A.

FIG. 8 illustrates a specific configuration example of the endoscope system in FIG. 7A.

FIG. 9A illustrates a configuration example of an optical fiber scanner.

FIG. 9B illustrates another configuration example of the optical fiber scanner.

FIG. 10A illustrates a modification of an oscillation mechanism.

FIG. 10B illustrates another modification of the oscillation mechanism.

### DESCRIPTION OF THE EMBODIMENTS

### First Embodiment

An illumination device and an illumination method according to a first embodiment of the present invention will now be described with reference to the drawings.

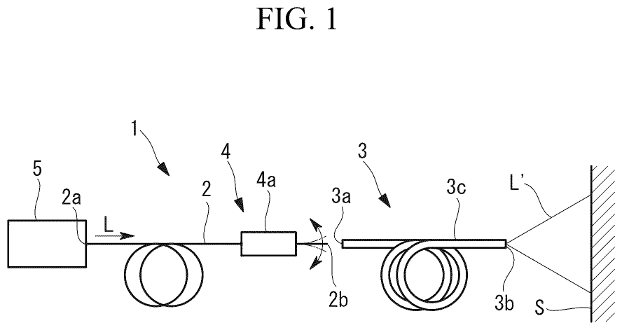

As shown in FIG. 1, an illumination device 1 according to this embodiment includes a first optical guide member 2, a second optical guide member 3, and an oscillation mechanism 4.

The first optical guide member 2 is a single-mode optical fiber, and a proximal end 2a of the first optical guide member 2 is connected to a light source 5. The light source 5 is a laser light source that outputs laser light L serving as coherent light. The illumination device 1 may further include the light source 5. The light L output from the light source 5 is optically guided through the optical fiber 2 from the proximal end 2a toward a distal end 2b, forms a point light source at the distal end 2b, and is output as diverging light from the distal end 2b.

The second optical guide member 3 is a multimode light guide and has an incidence surface 3a provided at the proximal end, an output surface 3b provided at the distal end, and a multimode propagation path 3c between the incidence surface 3a and the output surface 3b. For example, the light guide 3 is a single multimode optical fiber, and the propagation path 3c is a core of the optical fiber. The light guide 3 may be constituted of a plurality of multimode optical fibers. The incidence surface 3a is disposed facing the distal end 2b in the vicinity of the distal end 2b, and the light L output from the distal end 2b enters the propagation path 3c via the incidence surface 3a. The light L entering the propagation path 3c propagates through the propagation path 3c until reaching the output surface 3b, and is output from the output surface 3b toward a subject S. An illumination lens that adjusts the distribution of light may be disposed in front of the output surface 3b.

Because the light L is coherent, a speckle may occur due to interference between light beams scattered at the subject S. The oscillation mechanism 4 is a mechanism for reducing speckles and causes the light L incident on the incidence surface 3a and the incidence surface 3a to oscillate relatively in the radial direction of the incidence surface 3a.

In this embodiment, the oscillation mechanism 4 includes an optical fiber scanner 4a that scans the light L output from the distal end 2b by oscillating the distal end 2b of the optical fiber 2 in the radial direction of the optical fiber 2. The optical fiber scanner 4a may be of any type, such as a piezoelectric type using a piezoelectric element or an electromagnetic type using a permanent magnet and a coil. The optical fiber scanner 4a oscillates the distal end 2b at a predetermined frequency. The predetermined frequency is 10 Hz or higher, preferably 200 Hz or higher, and more preferably 3 kHz or higher. The optical fiber scanner 4a may scan the light L two-dimensionally along a predetermined scan trajectory. The scan trajectory may have any two-dimensional shape, such as a circular shape, an elliptical shape, a rectangular shape, a spiral shape, or a raster shape. The scan trajectory may alternatively have a one-dimensional shape.

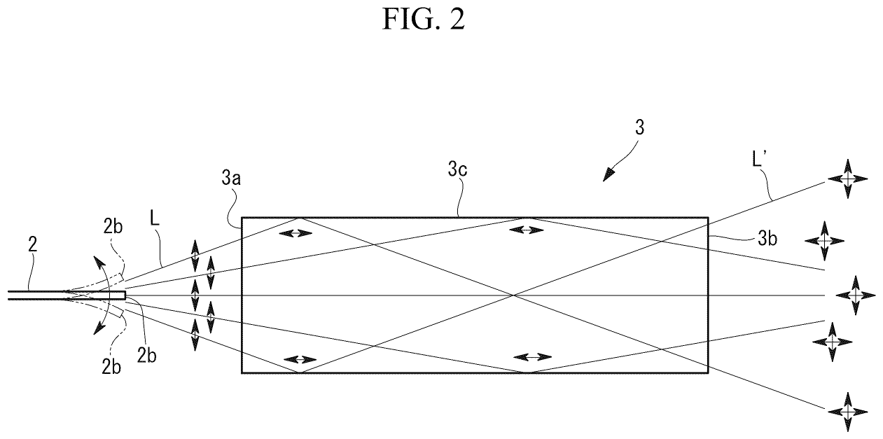

As shown in FIG. 2, the oscillation of the distal end 2b causes the light L output from the distal end 2b to oscillate in the radial direction of the incidence surface 3a, thus causing the incidence position and the incidence angle of the light L on the incidence surface 3a to change continuously and temporally. Accordingly, a speckle pattern, which will be described later, is uniformized and reduced.

In order to cause the light L output from the distal end 2b to enter the incidence surface 3a without loss, the oscillation amplitude of the distal end 2b, the core diameter of the optical fiber 2, and the effective diameter of the incidence surface 3a are designed such that the light L is scanned only within the effective diameter of the incidence surface 3a. Specifically, the effective diameter of the incidence surface 3a (i.e., the effective diameter of the light guide 3) is larger than the core diameter of the optical fiber 2. Moreover, the oscillation amplitude of the distal end 2b is smaller than the effective diameter of the incidence surface 3a, and the amplitude of the light L at the incidence surface 3a is smaller than the effective diameter of the incidence surface 3a.

The amplitude of the light L at the incidence surface 3a is estimated as h+dNA by using a distance d between the distal end 2b and the incidence surface 3a, an oscillation amplitude h of the distal end 2b, and a numerical aperture NA of the optical fiber 2. In order for the light L to enter the incidence surface 3a from the distal end 2b without loss, the amplitude of the light L may be smaller than or equal to an effective radius D/2 of the incidence surface 3a. Therefore, the oscillation amplitude h of the distal end 2b preferably satisfies Expression (1) indicated below.

$$h \leq D / 2 - dNA \qquad ( 1 )$$

On the other hand, if the oscillation amplitude h of the distal end 2b is too small, the speckle reduction effect decreases. Thus, the oscillation amplitude h preferably satisfies Expression (2) indicated below.

$$h \geq 0.1 \cdot D / 2 - dNA \qquad ( 2 )$$

In one design example, the distance d between the distal end 2b and the incidence surface 3a is 50 μm, the numerical aperture NA of the single-mode optical fiber constituting the optical fiber 2 is 0.1, the core diameter (effective diameter) of the multimode optical fiber constituting the light guide 3 is 250 μm, and the unilateral oscillation amplitude h of the distal end 2b is 50 μm.

Next, the operation of the illumination device 1 according to this embodiment will be described.

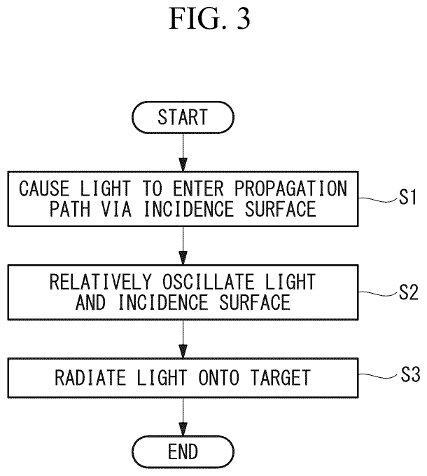

FIG. 3 illustrates the illumination method according to this embodiment using the illumination device 1. As shown in FIG. 3, the illumination method includes step S1 for causing the coherent light L from the light source 5 to enter the propagation path 3c via the incidence surface 3a, step S2 for relatively oscillating the light L incident on the incidence surface 3a and the incidence surface 3a so as to temporally change at least one of the incidence position and the incidence angle of the light L on the incidence surface 3a, and step S3 for radiating light L′ that has propagated through the propagation path 3c onto the subject (target) S.

In step S1, the light L output from the light source 5 enters the propagation path 3c via the optical fiber 2. In detail, the light L enters the optical fiber 2 via the proximal end 2a, is optically guided by the optical fiber 2 from the proximal end 2a to the distal end 2b, is output as diverging light from the distal end 2b, and enters the propagation path 3c via the incidence surface 3a.

Step S2 is executed concurrently with step S1. In step S2, the distal end 2b of the optical fiber 2 is oscillated by the oscillation mechanism 4, so that the incidence position or the incidence angle of the light L on the incidence surface 3a temporally changes at high speed.

Subsequently, in step S3, the light L′ that has propagated through the propagation path 3c is output from the output surface 3b toward the subject S, so as to illuminate the subject S.

In this case, in this embodiment, the light guide 3 has the multimode propagation path 3c, and the diverging light L including light beams of various angles enters the propagation path 3c via the incidence surface 3a. As shown in FIG. 2, the light beams included in the diverging light L propagate through the propagation path 3c while being reflected at different locations. Accordingly, the illumination light L′ constituted of a large number of light beams spatially multiplexed as a result of traveling along different optical path lengths is output from the output surface 3b of the light guide 3.

Furthermore, the incidence position and the incidence angle of the light L incident on the incidence surface 3a are temporally changed by the oscillation mechanism 4, so that the phase distribution of the illumination light L′ output from the output surface 3b is temporally multiplexed.

Accordingly, the illumination light L′ with the spatially and temporally multiplexed distribution is radiated onto the subject S, so that the speckle pattern is spatially and temporally uniformized. Consequently, speckles can be reduced.

Unlike the case where the intermediate positions of the optical fibers 2 and 3 are oscillated, as in Patent Literatures 1 and 2, the incidence position and the incidence angle of the light L on the incidence surface 3a are temporally changed in accordance with the oscillation of the distal end 2b of the optical fiber 2, so that the position and the angle of the light L propagating through the propagation path 3c can be temporally changed more dynamically. Consequently, a higher speckle reduction effect can be achieved.

When speckles are to be reduced by utilizing the oscillation of the light L, the speckle reduction effect normally appears at 10 Hz or higher. This is related to the frame rate of a normal individual imaging element. The speckle reduction effect becomes higher as the light L oscillates faster. The oscillation according to the method in Patent Literature 1 is normally at about 50 Hz, and the oscillation according to the method in Patent Literature 2 is normally at about 100 Hz to 200 Hz. In contrast, the optical fiber scanner 4a can readily achieve high-speed oscillation at 200 Hz or higher. Moreover, high-speed oscillation exceeding 3 kHz can also be achieved in accordance with resonant oscillation of the distal end 2b serving as a free end. Therefore, a high speckle reduction effect can be readily achieved.

According to the present invention, even in a case where laser speckles occur notably, as in a magnifying endoscope or a digital zoom display, such laser speckles, which are considered to be impossible to prevent in the related art, can be reduced by increasing the frequency of the optical fiber 2.

Moreover, by appropriately designing the effective diameter of the incidence surface 3a and the oscillation amplitude of the light L by the oscillation mechanism 4, the light L can be propagated from the light source 5 to the output surface 3b without loss. Therefore, the laser light L output from the light source 5 can be utilized for illuminating the subject S with a high efficiency value of approximately 100%, and the speckle pattern can be reduced without decreasing the illuminance.

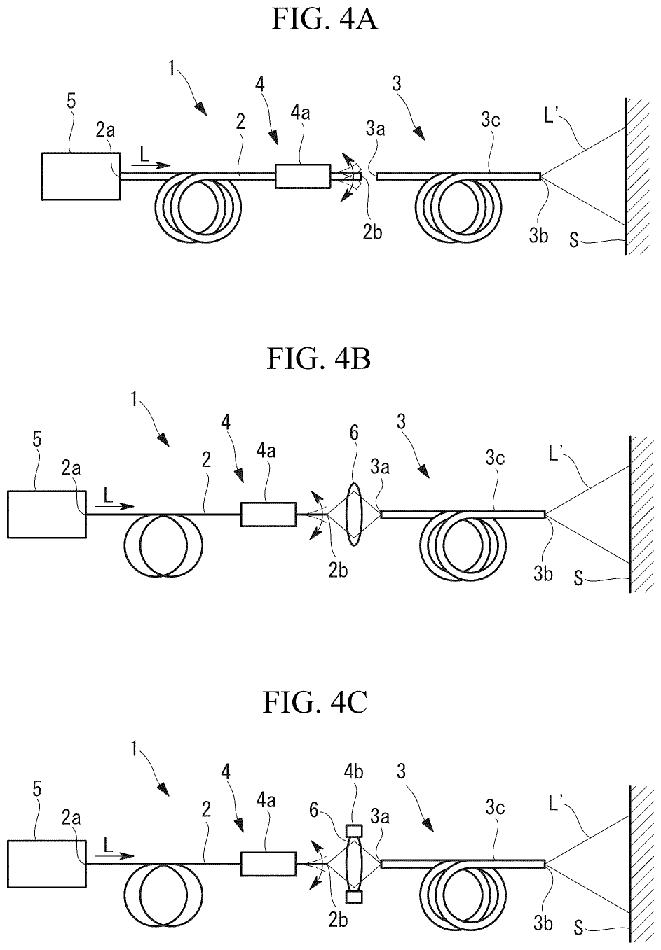

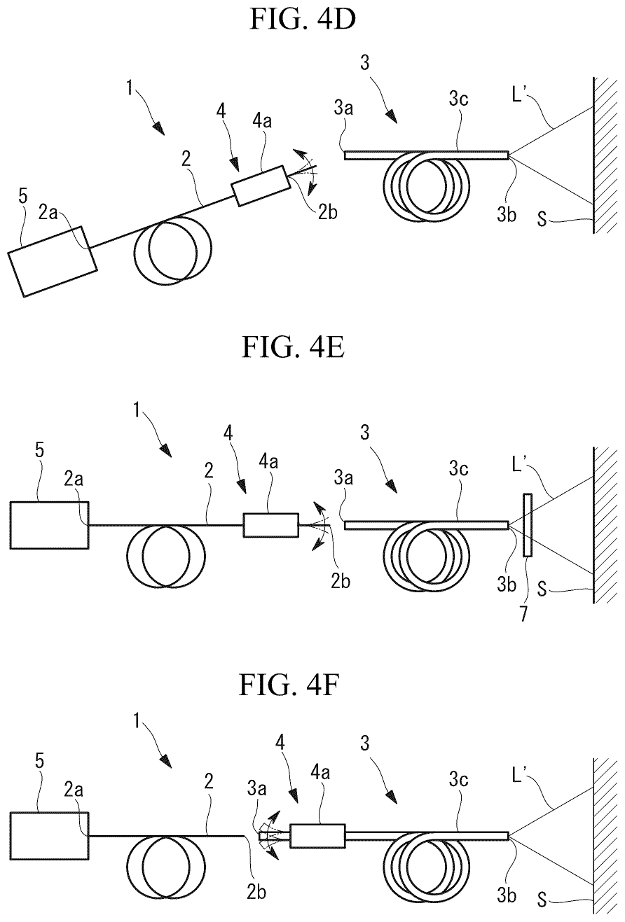

In this embodiment, the illumination device 1 is not limited to the above-described configuration and may be modified, as appropriate. FIGS. 4A to 4F illustrate modifications of the illumination device 1.

In the illumination device 1 in FIG. 4A, the first optical guide member 2 is a multimode optical fiber. By using the multimode type for both of the first optical guide member 2 and the second optical guide member 3, the light L from the light source 5 is further multiplexed. Accordingly, speckles can be further reduced.

In one design example of the illumination device 1 in FIG. 4A, the distance between the distal end 2b and the incidence surface 3a is 50 μm. In the multimode optical fiber serving as the optical fiber 2, the core diameter is 50 μm, the cladding diameter is 125 μm, and the numerical aperture is 0.22. In the multimode optical fiber serving as the light guide 3, the core diameter (effective diameter) is 500 μm, and the unilateral amplitude of the distal end 2b is 100 μm.

The illumination device 1 in FIG. 4B includes a relay optical system 6 between the first optical guide member 2 and the second optical guide member 3. The relay optical system 6 has at least one lens and focuses the diverging light L output from the distal end 2b onto the incidence surface 3a. The relay optical system 6 may have a mirror in place of the lens or in addition to the lens.

With the addition of the relay optical system 6, the degree of freedom in design, such as the distance between the first optical guide member 2 and the second optical guide member 3, can be enhanced. By adjusting the focusing angle of the light L by the relay optical system 6, the diverging angle of the illumination light L′ output from the output surface 3b can be increased. Furthermore, the connection efficiency of the light L between the distal end 2b and the incidence surface 3a can be optimized by the relay optical system 6, so that an occurrence of loss of the light L between the distal end 2b and the incidence surface 3a can be prevented more reliably. With the addition of the relay optical system 6, Expressions (1) and (2) indicated above may be satisfied by adjusting an image position d and an image height h of the distal end 2b formed by the relay optical system 6.

The illumination device 1 in FIG. 4C is a modification of the illumination device 1 in FIG. 4B. In addition to the optical fiber scanner 4a, the oscillation mechanism 4 includes an actuator 4b that oscillates the relay optical system 6 in a direction intersecting with the optical axis. The actuator 4b has, for example, a piezoelectric element and oscillates at least one lens included in the relay optical system 6. Accordingly, the light L incident on the incidence surface 3a receives oscillation by the relay optical system 6 in addition to the oscillation by the oscillation mechanism 4, so that speckles can be further reduced.

The modification in FIG. 4C may involve oscillating only the relay optical system 6 instead of oscillating both the distal end 2b and the relay optical system 6.

In the illumination device 1 in FIG. 4D, the optical axis of the first optical guide member 2 is inclined relative to the optical axis of the second optical guide member 3. With this arrangement, the propagation mode of the second optical guide member 3 is optimized, so that the intensity distribution of the illumination light L′ at the output surface 3b can be uniformized.

The illumination device 1 in FIG. 4E further includes a diffusing member 7 disposed in front of the output surface 3b of the second optical guide member 3. The diffusing member 7 is fixed relative to the output surface 3b and diffuses the illumination light L′ output from the output surface 3b. With the addition of the diffusing member 7, the speckle reduction effect can be further enhanced, and the intensity distribution of the illumination light L′ that illuminates the subject S can be further uniformized. Furthermore, since the diffusing member 7 is disposed at the side closest to the subject S and does not move, the illuminance of the illumination light L′ hardly decreases due to the diffusing member 7.

In the illumination device 1 in FIG. 4F, the oscillation mechanism 4 oscillates the incidence surface 3a at the proximal end of the second optical guide member 3 in the radial direction of the incidence surface 3a instead of the distal end 2b of the first optical guide member 2. Therefore, in step S2, the incidence position and the incidence angle of the light L on the incidence surface 3a are temporally changed in accordance with oscillation of the proximal end of the second optical guide member 3. Consequently, similar to the case where the distal end 2b is oscillated, the spatially and temporally multiplexed illumination light L′ can be radiated onto the subject S, so that speckles can be reduced. Since the oscillating second optical guide member 3 is not mechanically connected to the light source 5, the effect of the oscillation on the light source 5 can be eliminated.

The oscillation mechanism 4 may oscillate both of the distal end 2b and the incidence surface 3a. Consequently, speckles can be further reduced.

Next, an illumination device and an illumination method according to a second embodiment of the present invention will be described with reference to the drawings.

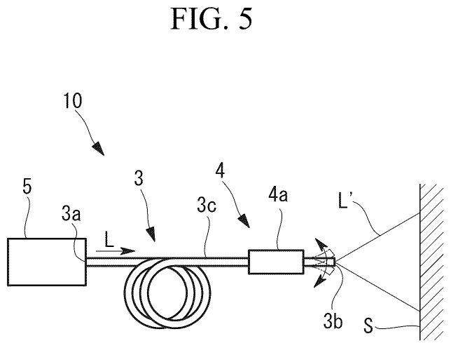

As shown in FIG. 5, an illumination device 10 according to this embodiment is different from that in the first embodiment in that the oscillation mechanism 4 oscillates the distal end of the light guide 3.

In this embodiment, components different from those in the first embodiment will be described, whereas components identical to those in the first embodiment will be given the same reference signs, and descriptions thereof will be omitted.

The illumination device 10 includes the multimode light guide 3 and the oscillation mechanism 4. The illumination device 10 may further include the light source 5.

As described in the first embodiment, the light guide 3 is constituted of one or multiple multimode optical fibers. The incidence surface 3a of the light guide 3 is connected to the light source 5. The light L output from the light source 5 enters the propagation path 3c via the incidence surface 3a, propagates through the propagation path 3c toward the output surface 3b, and is output as diverging light L′ from the output surface 3b.

Similar to the first embodiment, the oscillation mechanism 4 has the optical fiber scanner 4a. The optical fiber scanner 4a oscillates the distal end of the light guide 3 provided with the output surface 3b at a predetermined frequency in the radial direction of the light guide 3, thereby oscillating the light L′ output from the output surface 3b in the direction intersecting with the optical axis. The predetermined frequency is 10 Hz or higher, preferably 200 Hz or higher, and more preferably 3 kHz or higher. The optical fiber scanner 4a may be of any type, such as a piezoelectric type or an electromagnetic type.

In the illumination method according to this embodiment using the illumination device 10, the coherent light L from the light source 5 enters the multimode propagation path 3c via the incidence surface 3a (step S1′). Then, the light L′ that has propagated through the propagation path 3c is radiated onto the subject S via the output surface 3b (step S2′). Concurrently with steps S1′ and S2′, the distal end of the propagation path 3c provided with the output surface 3b is oscillated by the oscillation mechanism 4, whereby the position and the angle of the light L′ output from the output surface 3b temporally change (step S3′).

According to this embodiment, the light L propagates through the multimode propagation path 3c, so that illumination light L′ spatially multiplexed at the output surface 3b is generated. Moreover, the illumination light L′ output from the output surface 3b is oscillated, so that the illumination light L′ is temporally multiplexed. Accordingly, the illumination light L′ with the spatially and temporally multiplexed distribution is radiated onto the subject S, so that the speckle pattern is spatially and temporally uniformized. Consequently, speckles can be reduced.

Furthermore, since the first optical guide member 2 is not required in this embodiment, the number of components in the illumination device 10 can be reduced, as compared with the illumination device 1 according to the first embodiment.

### Third Embodiment

Next, an endoscope according to a third embodiment of the present invention will be described with reference to the drawings.

In this embodiment, components different from those in the first and second embodiments will be described, whereas components identical to those in the first and second embodiments will be given the same reference signs, and descriptions thereof will be omitted.

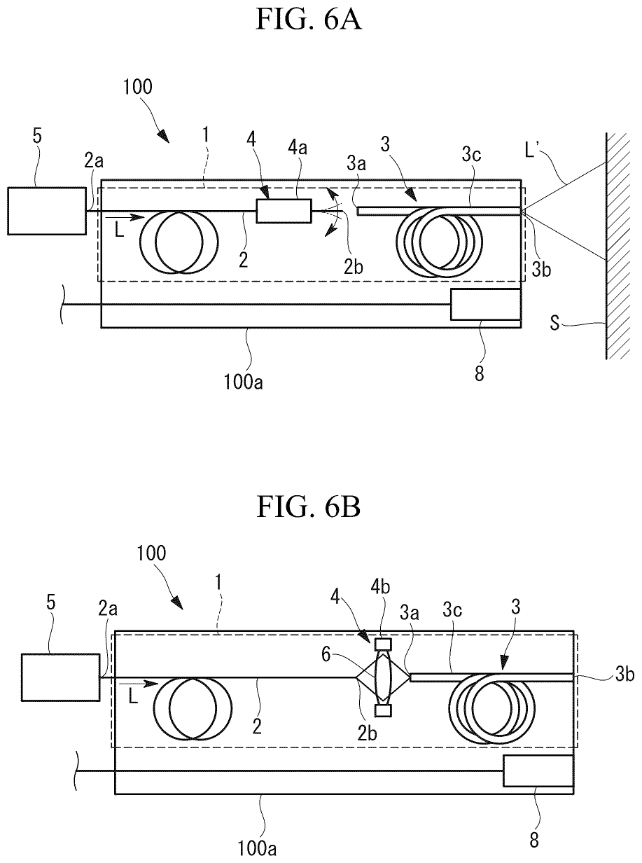

As shown in FIG. 6A, an endoscope 100 according to this embodiment includes the first optical guide member 2, the second optical guide member 3, the oscillation mechanism 4, and an imaging unit 8.

The first optical guide member 2, the second optical guide member 3, and the oscillation mechanism 4 constitute the illumination device 1 described in the first embodiment. The illumination device 1 is any one of the illumination devices 1 shown in FIG. 1 and FIGS. 4A to 4F. FIG. 6A illustrates the endoscope 100 equipped with the illumination device 1 in FIG. 1 as an example.

The illumination device 1 is provided inside a long insertion section 100a of the endoscope 100. The optical fiber 2 is disposed at the proximal end side of the insertion section 100a, and the light guide 3 is disposed at the distal end of the insertion section 100a.

The imaging unit 8 has, for example, an objective optical system and an imaging element. The imaging unit 8 captures an image of the subject S illuminated with the illumination light L′ output from the output surface 3b of the light guide 3, so as to acquire an endoscopic image.

In the endoscope 100 according to this embodiment, the illumination light L′ with the spatially and temporally multiplexed distribution is radiated onto the subject S, so that a speckle pattern generated at the subject S is spatially and temporally uniformized. Consequently, a high-quality endoscopic image with reduced speckles can be acquired by the imaging unit 8.

In this embodiment, as shown in FIG. 6B, the oscillation mechanism 4 may cause the light L to oscillate in accordance with oscillation of the relay optical system 6 instead of oscillation of the distal end 2b of the optical fiber 2. Specifically, the endoscope 100 may be equipped with the relay optical system 6 between the distal end 2b and the incidence surface 3a, and the oscillation mechanism 4 may be equipped with the actuator 4b.

### Fourth Embodiment

Next, an endoscope system according to a fourth embodiment of the present invention will be described with reference to the drawings.

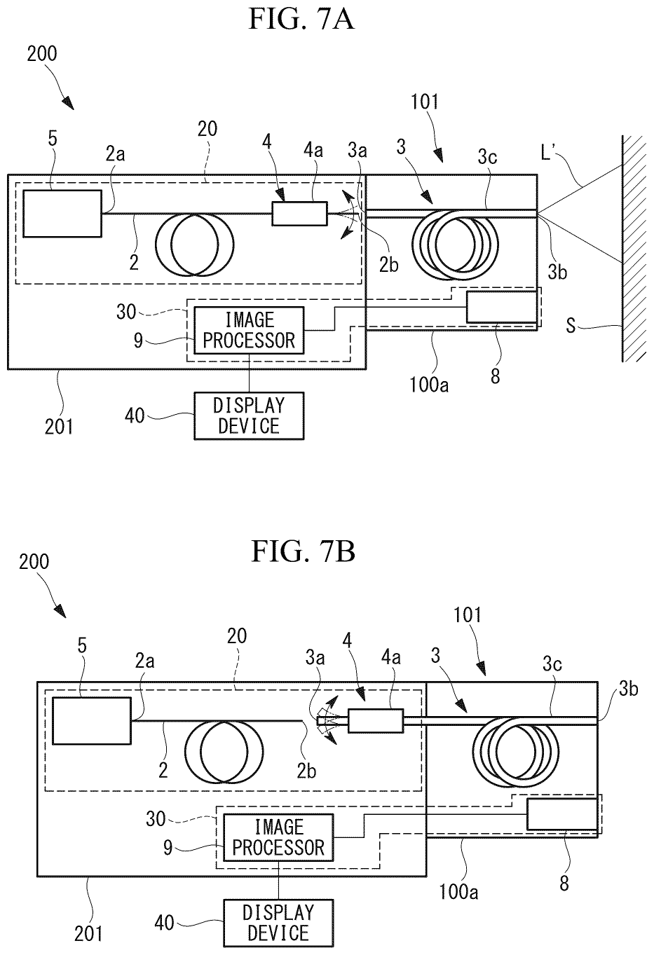

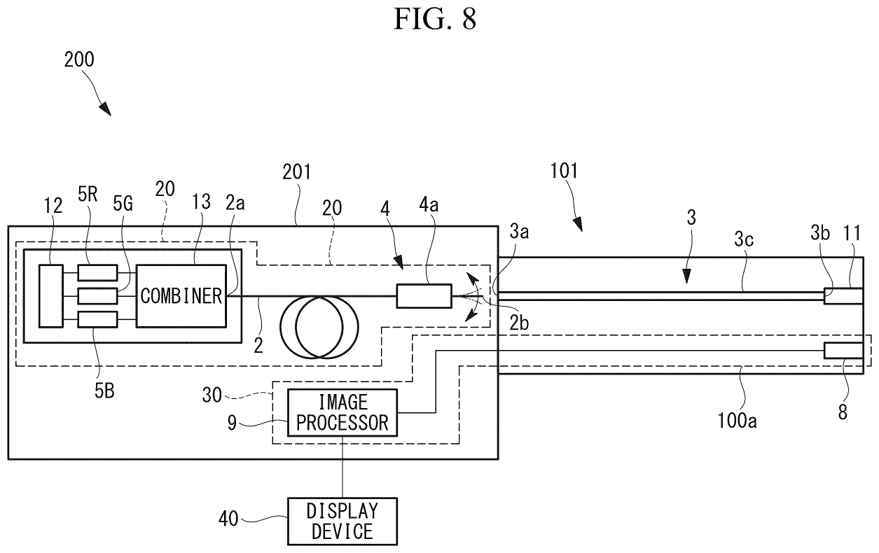

As shown in FIG. 7A, an endoscope system 200 according to this embodiment includes an endoscope 101, a light source device 20, an imaging device 30, and a display device 40. The endoscope system 200 also includes a housing 201 connected to the proximal end of the long insertion section 100a of the endoscope 101.

In this embodiment, components different from those in the first to third embodiments will be described, whereas components identical to those in the first to third embodiments will be given the same reference signs, and descriptions thereof will be omitted.

The endoscope 101 has the light guide 3. As described in the first embodiment, the light guide 3 is constituted of one or multiple multimode optical fibers and has the incidence surface 3a, the output surface 3b, and the multimode propagation path 3c. The light guide 3 is disposed within the insertion section 100a and extends in the longitudinal direction of the insertion section 100a. The incidence surface 3a is disposed at or in the vicinity of the proximal end surface of the insertion section 100a, and the output surface 3b is disposed at or in the vicinity of the distal end surface of the insertion section 100a. An illumination lens that adjusts the distribution of light may be disposed in front of the output surface 3b.

The light source device 20 is provided within the housing 201. The light source device 20 includes the first optical guide member 2, the oscillation mechanism 4, and the light source 5.

As described in the first embodiment, the first optical guide member 2 is a single-mode optical fiber. The proximal end 2a of the first optical guide member 2 is connected to the light source 5. The distal end 2b of the first optical guide member 2 is disposed at a position facing the incidence surface 3a. The light L output from the distal end 2b enters the propagation path 3c via the incidence surface 3a.

The oscillation mechanism 4 has the optical fiber scanner 4a that oscillates the distal end 2b.

The imaging device 30 has the imaging unit 8 provided at the distal end of the insertion section 100a, and also has an image processor 9 provided in the housing 201. An endoscopic image acquired by the imaging unit 8 is processed by the image processor 9 and is subsequently displayed on the display device 40.

The light source device 20 and the endoscope 101 may be detachably connected to each other. For example, the housing 201 may be provided with a first connector (not shown), the proximal end of the insertion section 100a may be provided with a second connector (not shown), and the light source device 20 and the endoscope 101 may be detachably connected to each other by using the first connector and the second connector.

FIG. 8 illustrates the configuration of the endoscope system 200 in more detail.

As shown in FIG. 8, the endoscope 101 may further include an illumination optical system 11 disposed at the distal end of the insertion section 100a. The illumination optical system 11 has a lens for widening the angle of the illumination light L′ and a fluorescent material excited by the illumination light L′. The illumination optical system 11 may also include the diffusing member 7 (see FIG. 4E) described in the first embodiment. The illumination light L′ output from the output surface 3b is radiated onto the subject S via the illumination optical system 11.

The light source device 20 includes at least one light source 5 and a light source driver 12 that drives the at least one light source 5. The light source 5 is a laser light source that outputs coherent laser light. In FIG. 8, the light source 5 provided includes three red, green, and blue semiconductor laser light sources 5R, 5G, and 5B. The light source device 20 may further include a combiner 13 that combines multiple light beams output from the multiple light sources 5R, 5G, and 5B.

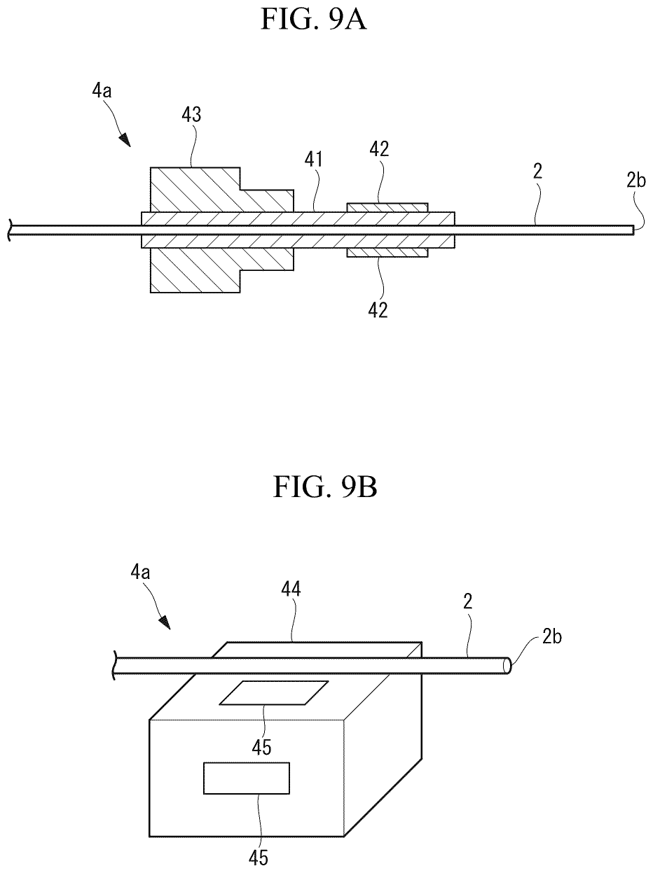

FIG. 9A illustrates a configuration example of the optical fiber scanner 4a of a piezoelectric type.

The optical fiber scanner 4a has a tubular ferrule 41 composed of an elastic material, at least one piezoelectric element 42 fixed to the outer peripheral surface of the ferrule 41, and a holder 43 fixed to the outer peripheral surface of the proximal end portion of the ferrule 41. The optical fiber 2 extends through the ferrule 41, and the ferrule 41 is fixed to the outer peripheral surface of the optical fiber 2. The holder 43 is fixed to an external member of the optical fiber scanner 4a, so that the ferrule 41 and the optical fiber 2 are supported in a cantilevered fashion. The piezoelectric element 42 receives an alternating voltage to undergo stretching vibration in the longitudinal direction of the optical fiber 2. The stretching vibration of the piezoelectric element 42 is transmitted to the optical fiber 2 via the ferrule 41. Consequently, bending vibration occurs at the distal end of the optical fiber 2 protruding from the distal end of the ferrule 41, thus causing the distal end 2b to oscillate.

FIG. 9B illustrates another configuration example of the optical fiber scanner 4a of the piezoelectric type. The optical fiber scanner 4a has a block 44 composed of an elastic material, and also has at least one piezoelectric element 45 fixed to the outer peripheral surface of the block 44. Although the block 44 shown in FIG. 9B has a rectangular parallelepiped shape, the block 44 may have any other shape, and may have a structure, such as a groove, for facilitating fixation of the optical fiber 2. The optical fiber 2 is fixed to a side surface, the bottom surface, or the top surface of the block 44 by using, for example, an adhesive, so that the optical fiber 2 is supported in a cantilevered fashion. The piezoelectric element 45 receives an alternating voltage to undergo stretching vibration in the longitudinal direction of the optical fiber 2. The stretching vibration of the piezoelectric element 45 is transmitted to the optical fiber 2 via the block 44. Consequently, bending vibration occurs at the distal end of the optical fiber 2, thus causing the distal end 2b to oscillate.

With the endoscope system 200 according to this embodiment, the illumination light L′ with the spatially and temporally multiplexed distribution is radiated onto the subject S, so that a speckle pattern occurring at the subject S is spatially and temporally uniformized. Consequently, a high-quality endoscopic image with reduced speckles can be acquired by the imaging unit 8.

The light source device 20 including the first optical guide member 2 and the oscillation mechanism 4 is disposed within the housing 201. A typical endoscope is normally equipped with the multimode second optical guide member 3, such as a light guide. Therefore, the illumination method according to this embodiment can be applied to the endoscope 101 without adding an optical system to the endoscope 101. Specifically, any of various endoscopes, such as a narrow endoscope or an endoscope not having a light scanning function, can be used as the endoscope 101.

With the light source device 20 and the endoscope 101 being detachable from each other, the light source device 20 can be used in combination with any endoscope 101 having the second optical guide member 3.

In this embodiment, the modifications described in the first embodiment may be applied to the endoscope system 200.

Specifically, the first optical guide member 2 may be a multimode optical fiber (see FIG. 4A).

The light source device 20 may include the relay optical system 6 between the distal end 2b and the incidence surface 3a (see FIGS. 4B and 4C). In this case, in place of or in addition to the optical fiber scanner 4a, the oscillation mechanism 4 may include the actuator 4b that oscillates the relay optical system 6 (see FIG. 4C).

The oscillation mechanism 4 may oscillate the incidence surface 3a at the proximal end of the second optical guide member 3 instead of the distal end 2b of the first optical guide member 2 (see FIG. 4F). As shown in FIG. 7B, the second optical guide member 3 may have a first section disposed within the insertion section 100a and including the output surface 3b and a second section disposed within the housing 201 and the including the incidence surface 3a. Accordingly, the oscillation mechanism 4 can be disposed within the housing 201. The first section and the second section are connected to each other in a separable manner by an optical connector (not shown), such as an optical fiber connector.

In the configuration in FIG. 7A, the incidence surface 3a may be disposed within the housing 201, and the light source device 20 and the endoscope 101 may be connected to each other by an optical connector, such as an optical fiber connector.

In this embodiment, the light L may be used as therapeutic light for treating tissue, such as a lesion. In that case, when treating the tissue, the oscillation of the light L may be stopped temporarily by stopping the operation of the oscillation mechanism 4.

In the first to fourth embodiments and the modifications thereof described above, the light guide 3 serving as the second optical guide member is constituted of one or multiple multimode optical fibers. Alternatively, the light guide 3 may be any other optical member that can transmit the light L in multiple modes. For example, the light guide 3 may be a fiber bundle or a multi-core fiber, or may be a linear glass rod.

In an embodiment where a single-mode fiber or a multimode fiber serving as the first optical guide member 2 disposed between the light guide 3 and the light source 5 is caused to oscillate, for example, the first optical guide member 2 may be inserted into a narrow target, such as a duct or lumen like the urinary duct or the pancreatic duct, and the distal end 2b thereof may be caused to oscillate. In this case, the length of the first optical guide member 2 serving as a point light source may be adjusted, so that the length of the second optical guide member 3 also functioning as a place for eliminating laser speckles can be appropriately changed.

In the first to fourth embodiments and the modifications thereof described above, the oscillation mechanism 4 includes the optical fiber scanner 4a and/or the actuator 4b. Alternatively, the oscillation mechanism 4 may oscillate the light L incident on the incidence surface 3a by any other means.

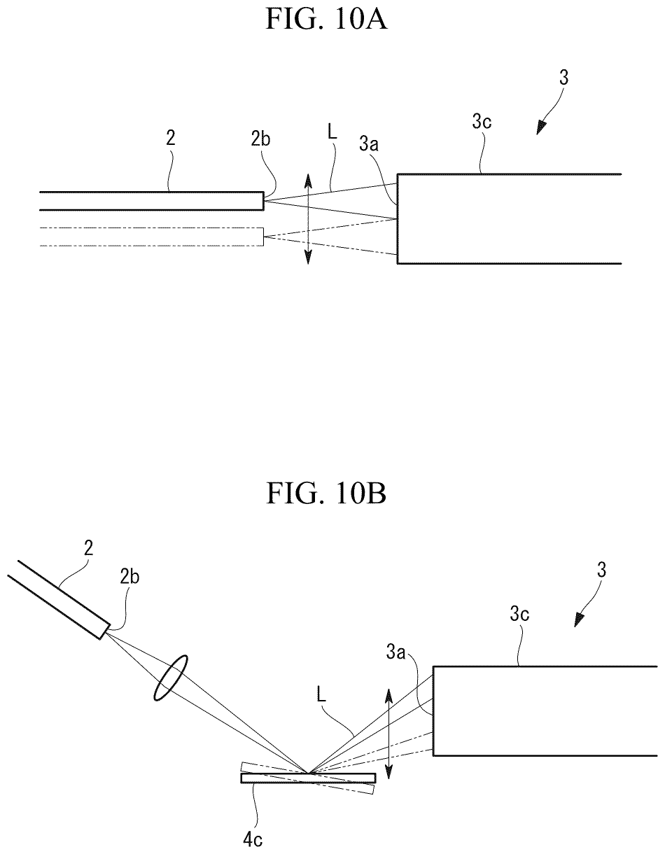

For example, as shown in FIG. 10A, the oscillation mechanism 4 may cause the distal end of the optical fiber 2 to move in a parallel fashion in the radial direction so as to oscillate the distal end 2b and the light L. Alternatively, as shown in FIG. 10B, the oscillation mechanism 4 may oscillate the light L in accordance with oscillation of a galvanometer mirror 4c.

### REFERENCE SIGNS LIST

1, 10 illumination device
2 first optical guide member (optical guide member), optical fiber
3 second optical guide member, light guide
4 oscillation mechanism
4a optical fiber scanner (scanner)
4b actuator
5 light source
6 relay optical system
7 diffusing member
8 imaging unit
20 light source device
100, 101 endoscope
200 endoscope system

1. An illumination method comprising:
causing coherent light from a light source to enter a multimode propagation path via an incidence surface;
relatively oscillating the light incident on the incidence surface and the incidence surface so as to temporally change at least one of an incidence position and an incidence angle of the light on the incidence surface; and
radiating the light that has propagated through the propagation path onto a target.

2. The illumination method according to claim 1,
wherein the light from the light source is caused to enter the propagation path via an optical guide member, and
wherein the relatively oscillating the light incident on the incidence surface and the incidence surface includes oscillating a distal end of the optical guide member.

3. The illumination method according to claim 1,
wherein the relatively oscillating the light incident on the incidence surface and the incidence surface includes oscillating a proximal end of the propagation path provided with the incidence surface.

4. An illumination method comprising:
causing coherent light from a light source to enter a multimode propagation path;
radiating the light that has propagated through the propagation path onto a target via an output surface; and
oscillating a distal end of the propagation path provided with the output surface so as to temporally change a position and an angle of the light output from the output surface.

5. The illumination method according to claim 1, wherein the light incident on the incidence surface is diverging light.

6. The illumination method according to claim 1, wherein a frequency of the oscillation is 10 Hz or higher.

7. The method according to claim 6, wherein the frequency of the oscillation is 200 Hz or higher.

8. An illumination device comprising:
a first optical guide member that comprises an optical fiber and that optically guides coherent light from a light source;
a second optical guide member that has an incidence surface, an output surface, and a multimode propagation path between the incidence surface and the output surface and that causes the light output from a distal end of the first optical guide member to enter the propagation path via the incidence surface; and
an oscillation mechanism that relatively oscillates the light incident on the incidence surface and the incidence surface so as to temporally change at least one of an incidence position and an incidence angle of the light on the incidence surface.

9. The illumination device according to claim 8, wherein the oscillation mechanism has a scanner that oscillates the distal end of the first optical guide member in a radial direction of the first optical guide member.

10. The illumination device according to claim 9, wherein an oscillation amplitude of the distal end of the first optical guide member is smaller than an effective diameter of the incidence surface.

11. The illumination device according to claim 9, further comprising:
a relay optical system disposed between the first optical guide member and the second optical guide member,
wherein the relay optical system focuses the light output as diverging light from the distal end of the first optical guide member onto the incidence surface of the second optical guide member.

12. The illumination device according to claim 8, wherein an optical axis of the first optical guide member is inclined relative to an optical axis of the second optical guide member.

13. The illumination device according to claim 8, further comprising a diffusing member that is disposed in front of the output surface of the second optical guide member, is fixed to the output surface, and diffuses the light.

14. An endoscope system comprising:
a light source device; and
an endoscope connected to the light source device,
wherein the light source device includes:
a light source;
a first optical guide member that comprises an optical fiber and that optically guides coherent light from the light source; and
an oscillation mechanism,
wherein the endoscope includes:
a second optical guide member that has an incidence surface, an output surface, and a multimode propagation path between the incidence surface and the output surface and that causes the light output from a distal end of the first optical guide member to enter the propagation path via the incidence surface, and
wherein the oscillation mechanism relatively oscillates the light incident on the incidence surface and the incidence surface so as to temporally change at least one of an incidence position and an incidence angle of the light on the incidence surface.

15. The endoscope system according to claim 14, wherein the oscillation mechanism has a scanner that oscillates the distal end of the first optical guide member in a radial direction of the first optical guide member.

16. The endoscope system according to claim 14, wherein the light source device and the endoscope are detachably connected to each other.
